(19) United States
(12) Patent Application Publication
(10) Pub. No.: US 2004/0028726 A1
(43) Pub. Date: Feb. 12, 2004

(54) Transdermal systems for the delivery of clonidine

(76) Inventors: Wilfried Fischer, Munchen (DE); Petra Huber, Munchen (DE)

Correspondence Address: FROMMER LAWRENCE & HAUG, 745 FIFTH AVENUE- 10TH FL., NEW YORK, NY, 10151, US

(21) Appl. No.: 10/616,248

(22) Filed: Jul. 9, 2003

Related U.S. Application Data
(63) Continuation of application No. 09/451,180, filed on Nov. 29, 1999, now abandoned.

(51) Int. Cl. A61K 9/70

(52) U.S. Cl. 424/449

(57) ABSTRACT

The invention relates to transdermal systems for the delivery of clonidine, characterised in that the clonidine is located in a contact adhesive layer based on a 2-ethylhexyl acrylate/vinyl acetate copolymer, and to their use in the treatment of hypertension, migraine, anxiety states, hyperkinetic behavioural disorders, withdrawal symptoms in alcohol or drug withdrawal and menopausal symptoms.

The invention relates to active-ingredient-containing transdermal systems (hereinafter referred to also as matrix patches or simply patches) for the delivery of clonidine and to their use in the treatment of hypertension, migraine, anxiety states, hyperkinetic behavioural disorders, withdrawal symptoms in alcohol or drug withdrawal and menopausal symptoms.

Active-ingredient-containing transdermal systems (“patches”) have been known to the person skilled in the field of pharmaceutical technology for about 20 years. They are divided essentially into two major technical systems: matrix systems and reservoir systems. The invention relates only to matrix systems in which medicinal active ingredients are embedded directly in a semi-solid matrix of polymers.

Clonidine is an anti-sympathicotonic agent having an imidazoline structure. It has affinity for α1-adrenoceptors and—more strongly—for pre- and post-synaptic α2-adrenoceptors and lowers peripheral sympathetic tone. Clonidine brings about especially a lowering of blood pressure by virtue of decreasing cardiac output and—in the case of prolonged medication—by reducing peripheral vascular resistance. At the same time it reduces the release of renin with a decrease in angiotensin II in the blood plasma, with aldosterone being released from the adrenal cortex.

Clonidine is used, for example, in the following indications:

hypertension

migraine

anxiety states

hyperkinetic behavioural disorders

withdrawal symptoms in alcohol or drug withdrawal

menopausal symptoms

Clonidine hydrochloride exists in the form of a mesomeric component. The chemical name is 2-(2,6-dichlorophenylamino)-2-imidazoline hydrochloride. Molecular formula: C₉H₉Cl₂N₃.HCl, molecular weight: 266.56.

Various transdermal systems that contain clonidine have been developed. For example, U.S. Pat. No. 4,559,222 of Dec. 17, 1985 describes a multi-layer transdermal system in which clonidine base is contained in mineral oil together with colloidal silicon dioxide in a first layer in a polyisobutylene adhesive. To that layer there is applied a microporous membrane, to which, in turn, a layer of adhesive is applied. That adhesive layer is affixed to the skin. The transdermal system is covered on the side of the active-ingredient-containing layer with a film that is impermeable to clonidine. Disadvantages of that system are the known poor skin tolerability of polyisobutylene adhesives, the complicated and expensive manufacturing process arising from the large number of layers that are required and the fundamental physical instability of the system, because the layer coming into contact with the skin becomes saturated with clonidine in the course of storage, with the result that the release behaviour of the system changes, that is to say a system that has been stored for a prolonged period will, after being affixed to the skin, release the active ingredient from the contact layer at a faster rate than further active ingredient can be supplied through the microporous membrane. A further disadvantage is the poor adhesive strength of the system. Since the transdermal system is intended to be worn for a period of seven days, the manufacturer must also provide an active-ingredient-free plaster that has to be affixed over the actual clonidine-containing system in order to provide a reliable fixing. This further increases the costs as well as the effort required of the user.

U.S. Pat. No. 5,762,952 of Jun. 9, 1998 describes an improved system, consisting of a self-crosslinking acrylate adhesive into which e.g. clonidine is incorporated together with auxiliaries, such as solvents or absorption promoters, that are volatile at relatively high temperatures. The crosslinking is necessary in order to increase the consistency of the adhesive substance, which is greatly reduced by the addition of large amounts of liquid components, such as solvents or absorption promoters, to such an extent that a coherent layer of adhesive is no longer obtained. Disadvantages of that invention are the use, firstly, of toxic crosslinking agents and also of solvents and absorption promoters that are potentially irritating to the skin.

U.S. Pat. No. 5,958,446 describes an invention according to which a mixture of self-adhesive acrylates and polyisobutylene or silicones give a higher flow rate through the skin than when the polymers are used alone. Although the patent claims the use of clonidine as active ingredient, it does not give an example thereof. The disadvantage of the invention described is that the combination of two polymers in the majority of the described examples (e.g. with 17β-oestradiol, norethisterone acetate, pilocarpine, all substances having good penetration through the skin) was produced using absorption promoters such as lecithin or propylene glycol in order to obtain an adequate flow rate. That is to say, the use of the mixtures of polymers described in the patent is not sufficient on its own to produce transdermal systems having a satisfactory action.

The aim of the present invention is to provide a transdermal system for the delivery of clonidine that is very economical to produce, is very kind to the skin, is easy for the patient to use, does not require additional fixing aids, releases from 100 to 300 μg of clonidine through the skin per day and does not contain any toxic crosslinking agents or solvents/absorption promoters.

According to the invention, that problem is solved by a transdermal system for the delivery of clonidine in accordance with patent claim 1.

The invention accordingly relates to transdermal systems for the delivery of clonidine that are characterised in that they have a clonidine-containing contact adhesive layer based on a 2-ethylhexyl acrylate/vinyl acetate copolymer.

The invention relates also to the use of such transdermal systems in the treatment of hypertension, migraine, anxiety states, hyperkinetic behavioural disorders, withdrawal symptoms in alcohol or drug withdrawal and menopausal symptoms in accordance with patent claim 15.

The invention is based on the surprising finding that a pressure-sensitive acrylate-based contact adhesive which consists exclusively of the monomers 2-ethylhexyl acrylate and vinyl acetate fulfils all the above-mentioned requirements: clonidine base is soluble in an adequate concentration in the dried adhesive and the chemical potential of the clonidine in the dried adhesive is high enough, without the admixture of further components, to maintain an adequate flow of active ingredient through intact skin over a period of seven days. The adhesive requires no crosslinking agent to produce an optimum consistency in combination with the clonidine dissolved therein. The adhesive strength is so high that excellent adhesion is achieved over a period of seven days without significant irritation to the skin. The use of additional fixing aids is superfluous.

Further advantageous and preferred embodiments are the subject of the subsidiary claims.

An advantageous embodiment is characterised in that the contact adhesive layer comprises clonidine in a concentration range of from 0.1 to 20% by weight.

A further advantageous embodiment is characterised in that the contact adhesive layer comprises clonidine in a concentration range of from 2 to 10% by weight.

A further advantageous embodiment is characterised in that in addition to comprising the clonidine and the 2-ethylhexyl acrylate/vinyl acetate copolymer, the contact adhesive layer also comprises fillers and/or skin-protective substances and/or tackifiers.

A further advantageous embodiment is characterised in that the clonidine-containing contact adhesive layer forms a layer of a planar self-adhesive patch of multi-layered structure.

A further advantageous embodiment is characterised in that in addition to having the clonidine-containing contact adhesive layer, the patch also has a covering and, on the side opposite from the covering, a removable support that temporarily covers the contact adhesive layer.

A further advantageous embodiment is characterised in that the covering consists of plastics film, plastics foam, woven fabric or non-woven fabric.

A further advantageous embodiment is characterised in that the support consists of plastics film or paper or a laminate thereof.

A further advantageous embodiment is characterised in that the support is siliconised.

A further advantageous embodiment is characterised in that the plastics film is polyester, polyethylene or polypropylene film.

A further advantageous embodiment is characterised in that the dry contact adhesive layer has a weight per unit area of from 20 to 150 g/m².

A further advantageous embodiment is characterised in that the dry contact adhesive layer has a weight per unit area of from 50 to 120 g/m².

A further advantageous embodiment is characterised in that the delivery rate is from 10 to 1000 μg of clonidine per day.

A further advantageous embodiment is characterised in that the delivery rate is from 50 to 500 μg of clonidine per day.

The invention is described in greater detail below but is not limited thereby.

The production of clonidine patches is carried out on conventional machines which will be known to a person skilled in the art.

Clonidine base is dissolved or dispersed in a suitable, readily volatile solvent, e.g. ethyl acetate, ethanol or isopropanol. The solution/dispersion is mixed with a solution of the pressure-sensitive contact adhesive described above in a suitable vessel. Customary substances such as fillers, skin-protective substances, tackifiers or the like may be added if desired, but it is not essential. The mixture of clonidine and the acrylate and optionally further substances is applied to a substrate or support, for example of siliconised plastics films, siliconised paper or the like, in a customary coating machine and freed of solvent in a dryer located downstream. After leaving the dryer, the then dry and self-adhesive active ingredient/adhesive matrix is laminated with a further layer, which may be e.g. a plastics film, a non-woven fabric, a plastics foam, a woven fabric or the like, for covering purposes.

In a further processing step, in a cutting or punching device known to a person skilled in the art the desired transdermal systems of a defined shape and size are cut or punched out. The finished systems are introduced into sachets or similar packagings for protection purposes.

The systems typically contain clonidine in a concentration range of from 0.1 to 20%, preferably in the range of from 2 to 10%. The weight per unit area of the dried contact adhesive layer (matrix) is usually in the range of from 20 to 150 g/m², preferably in the range of from 50 to 120 g/m² The delivery rate is in the range of from 10 to 1000 μg of clonidine per day, preferably in the range of from 50 to 500 μg per day.

For the characterisation of the transdermal systems in respect of their delivery of active ingredient, essentially two methods are employed:

1. in vitro skin permeation tests

2. in vitro release tests in accordance with current pharmacopoeias.

Skin permeation tests are frequently carried out on isolated skin from nude mice. In such tests a piece of patch is affixed to the upper side of the skin and mounted in a diffusion cell. A buffer solution (acceptor) comes into contact with the underside of the skin and the time-dependent change in concentration in the acceptor medium is measured.

The results obtained using the preparations according to the invention are given in the following Examples.

The in vitro release tests are carried out in glass vessels constructed as stipulated in the pharmacopoeias. In a cylindrical round-bottomed vessel of 1 litre capacity, the patch is attached to a perforated plate so that the adhesive layer faces upwards. The perforated plate is placed on the bottom of the vessel and the vessel is filled with water, whereupon stirring is carried out with a defined stirrer until concentration equilibrium is obtained. In these tests the time-dependent concentration in the medium into which the release is effected is likewise measured. The results of the tests are given in the Examples.

The difference between these methods is that the release tests take account only of the release behaviour of the active ingredient from the patch, which does not, however, generally correlate with the biological action, whereas the skin permeation model, in addition to giving consideration to the necessary release, also takes account of the distribution of the active ingredient into the skin and the diffusion through the skin. Using that method, correlations with the biological action are generally possible.

The following Examples illustrate, but do not limit, the invention.

### COMPARATIVE EXAMPLE 1

A commercially available clonidine patch, Catapres® TTS, having the following characteristics:

| | |
|---|---|
| clonidine content: | 5 mg |
| surface area: | 7 cm² |

Composition (Qualitative):

mineral oil

polyisobutylene

colloidal silicon dioxide

microporous polypropylene membrane

was subjected to an in vitro dissolution test in accordance with the European Pharmacopoeia. The results are shown in Table 1.

In addition, the in vitro skin permeation behaviour was investigated using a mouse skin model.

Procedure:

A 1.5 cm² piece of skin, which has been freed of subcutaneous tissue, from a female nude mouse is placed over the opening, exactly 1 cm² in size, of an automatic diffusion cell, affixed with an approximately 1.5 cm² piece of the clonidine patch and sealed on the cell with a pressing device. The cell is then filled with 25 ml of a physiological HEPES buffer solution and the temperature is maintained at 34° C. At defined intervals, samples are taken from the buffer solution and their active ingredient content is determined by high pressure liquid chromatography.

All the patches described below were tested in accordance with that procedure.

The results are shown in Table 2.

### COMPARATIVE EXAMPLE 2

For comparison purposes, a clonidine patch was prepared using a self-crosslinking acrylate adhesive without added absorption promoters. The system had the following characteristics:

| | | |
|---|---|---|
| clonidine content: | 5.25 | mg |
| surface area: | 7 | cm² |
| Composition: | | |
| acrylate adhesive Duro-Tak 87-2052 | 64.75 | mg |
| siliconised polyester film FL2200075 1S** | 7 | cm² |
| polyester film Hostaphan MN 19 MED*** | 7 | cm² |

The Duro-Tak contact adhesive becomes self-crosslinking at low temperature by the addition of aluminium acetylacetonate.
*National Starch & Chemical, Zutphen, Netherlands
**Rexam, Apeldoorn, Netherlands
***Mitsubishi Polyester Foils, Frankfurt, Germany

Preparation:

Clonidine is dissolved in ethyl acetate. The solution is added to a sufficient amount of the commercially available adhesive solution and homogenised using a stirrer. Using a doctor blade, the homogeneous solution is then spread in a defined layer thickness onto a sheet of a siliconised polyester film (about 75 μm). For the purpose of drying and crosslinking, the sheet is then dried in a drying cabinet at 50° C. for 30 minutes. An approximately 19 μm thick polyester film is then laminated onto the adhesive layer. Patches 7 cm² in size are punched out of the finished laminate using a hand punch.

Skin Permeation:

see Comparative Example 1

The results are shown in Table 2.

### EXAMPLE

A clonidine patch according to the invention has the following characteristics:

| | | |
|---|---|---|
| clonidine content: | 5.25 | mg |
| surface area: | 7 | cm² |
| Composition: | | |
| acrylate adhesive Duro-Tak 87-4098 | 64.75 | mg |
| siliconised polyester film FL200075 1S** | 7 | cm² |
| polyester film Hostaphan MN 19 MED*** | 7 | cm² |

Preparation:

see Comparative Example 2

In Vitro Release:

see Comparative Example 1

The results are shown in Table 1.

Skin Permeation:

see Comparative Example 1

The results are shown in Table 2.

TABLE 1
In vitro release clonidine transdermal system
(matrix patch)

| | | |
|---|---|---|
| Time | Comp. Ex. 1 | Example 1 |

| | |
|---|---|
| (hours) | Release of clonidine (%) |

| | | |
|---|---|---|
| 2 | 10.44 | 9.96 |
| 4 | 11.82 | 13.92 |
| 24 | 20.95 | 19.76 |

TABLE 2
In vitro skin permeation clonidine transdermal
system (matrix patch)

| Time | Comp. Ex. 1 | Comp. Ex. 2 | Example |
|---|---|---|---|
| (hours) | Clonidine permeation (μg/cm²) | | |
| 3 | 24 | | 24.5 |
| 6 | 56 | | 54 |
| 9 | 80.5 | | 86 |
| 14 | 113.5 | 23.32 | 128.5 |
| 19 | 139 | 46.94 | 165.5 |
| 24 | 163.5 | 55.84 | 186 |
| 32 | | 82.68 | |
| 36 | 233.5 | | 229 |
| 40 | | 105.37 | |
| 48 | 305.5 | | 259.5 |

1. Transdermal system for the delivery of clonidine, characterised in that it comprises a clonidine-containing contact adhesive layer based on a 2-ethylhexyl acrylate/vinyl-acetate copolymer.

2. Transdermal system according to claim 1, characterised in that the contact adhesive layer comprises clonidine in a concentration range of from 0.1 to 20% by weight.

3. Transdermal system according to claim 2, characterised in that the contact adhesive layer comprises clonidine in a concentration range of from 2 to 10% by weight.

4. Transdermal system according to any one of claims 1 to 3, characterised in that in addition to comprising the clonidine and the 2-ethylhexyl acrylate/vinyl acetate copolymer, the contact adhesive layer also comprises fillers and/or skin-protective substances and/or tackifiers.

5. Transdermal system according to any one of the preceding claims, characterised in that the clonidine-containing contact adhesive layer forms a layer of a planar self-adhesive patch of multi-layered structure.

6. Transdermal system according to claim 5, characterised in that in addition to having the clonidine-containing contact adhesive layer, the patch also has a covering and, on the side opposite from the covering, a removable support that temporarily covers the contact adhesive layer.

7. Transdermal system according to claim 6, characterised in that the covering consists of plastics film, plastics foam, woven fabric or non-woven fabric.

8. Transdermal system according to claim 6, characterised in that the support consists of plastics film or paper or a laminate thereof.

9. Transdermal system according to claim 8, characterised in that the support is siliconised.

10. Transdermal system according to claim 7 or 8, characterised in that the plastics film is polyester, polyethylene or polypropylene film.

11. Transdermal system according to any one of claims 5 to 10, characterised in that the dry contact adhesive layer has a weight per unit area of from 20 to 150 g/m².

12. Transdermal system according to claim 11, characterised in that the dry contact adhesive layer has a weight per unit area of from 50 to 120 g/m².

13. Transdermal system according to any one of the preceding claims, characterised in that the delivery rate is from 10 to 1000 μg of clonidine per day.

14. Transdermal system according to claim 13, characterised in that the delivery rate is from 50 to 500 μg of clonidine per day.

15. Use of a transdermal system according to any one of the preceding claims in the treatment of hypertension, migraine, anxiety states, hyperkinetic behavioural disorders, withdrawal symptoms in alcohol or drug withdrawal and menopausal symptoms.
